(19) United States
(12) Patent Application Publication
(10) Pub. No.: US 2026/0250588 A1
(43) Pub. Date: Aug. 27, 2026

(54) BIOGAS FEED FOR CARBON MONOXIDE PRODUCTION

(71) Applicant: Topsoe A/S, Kgs. Lyngby (DK)

(72) Inventors: Martin ØSTBERG, Tune (DK); Peter Mølgaard MORTENSEN, Roskilde (DK); Troels Juel FRIIS-CHRISTENSEN, Smørum (DK)

(73) Assignee: Topsoe A/S, Kgs. Lyngby (DK)

(21) Appl. No.: 18/874,697

(22) PCT Filed: Jun. 15, 2023
(86) PCT No.: PCT/EP2023/066147

§ 371 Date: Dec. 13, 2024

(30) Foreign Application Priority Data
Jun. 17, 2022 (EP) 22179669.1

(51) Int. Cl. C10K 1/00

(52) U.S. Cl. CPC C10K 1/005

(57) ABSTRACT

The present invention relates to a carbon monoxide plant and process for effective use of biogas. The carbon monoxide plant comprises: a first biomass feed, a biomass digester, a reformer section, a CO₂-removal section and a cryogenic separation section. A process for providing a CO-rich product stream from a first biomass feed, using the plant of the invention, is also provided.

### TECHNICAL FIELD

The present invention relates to a carbon monoxide plant and process for effective use of biogas.

### BACKGROUND

Biogas is a renewable energy source that can be used for heating, electricity, and many other operations. Biogas can be cleaned and upgraded to natural gas standards, to become bio-methane. Biogas is considered to be a renewable resource because its production-and-use cycle is continuous, and it generates no net carbon dioxide. When the organic material has grown, it is converted and used. It then regrows in a continually repeating cycle. From a carbon perspective, as much carbon dioxide is absorbed from the atmosphere in the growth of the primary bio-resource as is released, when the material is ultimately converted to energy.

Biogas is a mixture of gases produced by the breakdown of organic matter in the absence of oxygen. Biogas can be produced from raw materials such as agricultural waste, manure, municipal waste, plant material, sewage, green waste or food waste. Biogas is primarily methane (CH₄) and carbon dioxide (CO₂) and may include small amounts of hydrogen sulfide (H₂S), moisture, siloxanes, and possibly other components.

It is feasible to produce synthesis gas (syngas) from biogas feed. However, the syngas produced from a biogas feed will typically contain a substantial amount of CO and remaining CO₂ as a consequence of the high CO₂ content in the biogas. This results in a synthesis gas with an H₂/CO ratio lower than is typically required for e.g. FT synthesis or MeOH synthesis.

Cryogenic separation processes are typically driven by the Joule-Thomsen effect. This means essentially that—at a given temperature and pressure—expansion of a gas or liquid results in cooling of the medium. Like in a refrigerator, this means that cooling can be achieved by first compressing a gas and subsequently expanding it.

Cryogenic plants are today designed as standalone units, where cooling inside the unit is supplied by a dedicated compressor. In a CO cold box, cooling is provided by recycling CO in large amounts, while CO₂ separation is driven by expanding the CO₂ product. Cryogenic separation processes can also be expensive and resource-intensive, because large compressors are needed to drive the cooling mechanism in the separation sections. Known technology includes US2013/0111948 A1.

There is a need for improved chemical plants and processes, in which the potential for synergy between the various units is optimised. A better utilisation of the components of a biogas feed is also required.

### SUMMARY

It has been found by the present inventor(s) that biogas synthesis followed by synthesis gas production is very attractive if CO production is the main purpose of the synthesis gas unit. The present inventors have therefore provided an efficient way to utilize the components of the biogas, involving separation of the biogas into its constituent components.

So, in a first aspect the present invention relates to a carbon monoxide plant, said plant comprising:
a first biomass feed,
a biomass digester, arranged to receive the first biomass feed, and provide a biogas stream,
a reformer section arranged to receive at least a portion of the biogas stream, and provide a first synthesis gas stream,
a CO₂-removal section arranged to receive a synthesis gas stream from the reformer section and provide a CO₂-rich stream and a CO₂-depleted stream;
a cryogenic separation section, arranged to receive a CO₂-depleted stream from the CO₂-removal section and provide at least a CO-rich product stream.

A process is also described for providing a CO-rich product stream from a first biomass feed, in a plant as described herein, said process comprising the steps of:
supplying a first biomass feed to the biomass digester, and providing a biogas stream,
feeding at least a portion of the biogas stream to the reformer section, and providing a first synthesis gas stream,
feeding at least a portion of a synthesis gas stream from the reformer section to a CO₂-removal section and providing a CO₂-rich stream and a CO₂-depleted stream;
feeding the CO₂-depleted stream from the CO₂-removal section to the cryogenic separation section, and providing at least a CO-rich product stream.

Further details of the technology are provided in the enclosed dependent claims, FIGURES and examples.

### FIGURES

The technology is illustrated by means of the following schematic illustrations, in which:

FIG. 1 shows a schematic process layout of the plant of the invention.

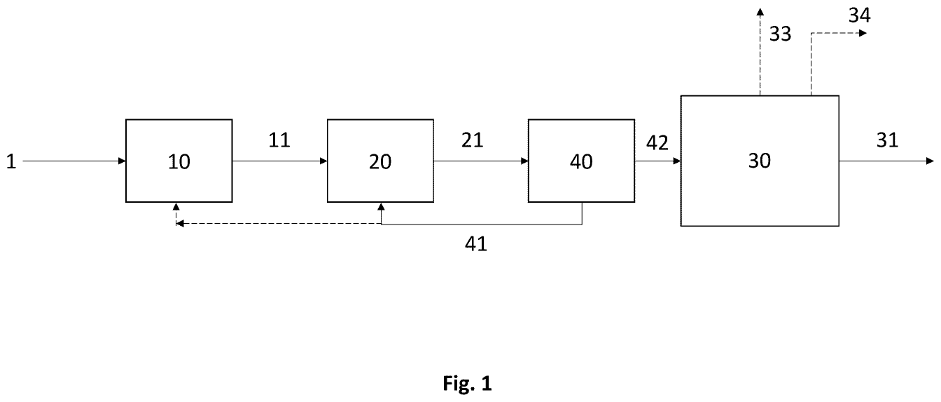

### DETAILED DISCLOSURE

Unless otherwise specified, any given percentages for gas content are % by volume. All feeds are preheated as required.

The term “synthesis gas” (abbreviated to “syngas”) is meant to denote a gas comprising hydrogen, carbon monoxide, carbon dioxide and small amounts of other gasses, such as argon, nitrogen, methane, etc.

As noted above, syngas produced from biogas feed will typically contain a substantial amount of CO and remaining CO₂ as a consequence of the high CO₂ content in the biogas. This results in a synthesis gas with a lower H₂/CO ratio lower than is typically required for e.g. FT synthesis or MeOH synthesis. This makes it very attractive to be used if CO production is the main purpose of the synthesis gas unit. The present inventors have therefore provided an efficient way to utilize the components of the biogas, involving separation of the biogas into its constituent components.

In a CO production unit, a cold box (cryogenic separation) will be used to make the concentrated stream of CO gas. Other contents in the syngas include hydrogen, methane and carbon dioxide. The latter can either be a stream from the cryogenic separation or it may be washed out prior to the cryogenic separation. However, it is often economical favorable to include it in the “cold box”. The by product streams can be utilized if not desired as additional product streams from the plant. Methane will typically be recycled to the feed, carbon dioxide and hydrogen can be utilized in a reverse water gas shift reactor (rWGS) forming additional CO:

$$C {O}_{2} + {H}_{2} \text{?} CO + {H}_{2} O$$

The product from the rWGS can be mixed with the raw synthesis gas and separate in the same “cold box”. CO₂ may also be recycled upstream either to the feed if carbon limits for the main reformer allows or used in an additional synthesis gas reactor (APOC or electrical post converter) again to increase the CO production. Then hydrogen will typically be utilized as a separate product.

A carbon monoxide plant is therefore provided, said plant comprising:
a first biomass feed,
a biomass digester,
a reformer section
a CO₂-removal section
and a cryogenic separation section.

### Biomass Feed

A biomass feed is typically a liquid slurry, with a total solids content of between 20-40%. Apart from water, biomass principally comprises organic material which can be converted by the action of microbes to a biogas, e.g. in an anaerobic digestion with anaerobic organisms or methanogen inside an anaerobic digester. Sources of biomass feed include agricultural waste, such as manure, sewage, green waste and food waste, as well as industrial waste e.g. from food or drink production.

Apparatus for handling and supply of the biomass feed to the plant are known to the skilled engineer.

### Biomass Digester

A biomass digester is arranged to receive the first biomass feed and provide a biogas stream. The term “biogas” in connection with the present invention denotes a gas with the following composition:

| Compound | % |
|---|---|
| Methane | 50-75 |
| Carbon dioxide | 25-50 |
| Nitrogen | 0-10 |
| Hydrogen | 0-1 |
| Oxygen | 0-1 |

The bacteria which convert the biomass feed into biogas are capable of digesting most hydrocarbon feedstocks. This is important in the combination of a biogas unit with a chemical synthesis unit.

A biomass digester is typically in the form of a pressure reaction vessel with appropriate inlet(s) for biomass and outlet(s) for biogas. Additional inlets and outlets may be provided for the various waste water streams recycled according to the invention. Inlets and outlets may also be provided for e.g. sampling the contents of the digester or introducing or removing microbial matter.

The biomass digester operates most effectively at around 50° C. In one aspect, the plant comprises means for heating the biomass digester, preferably a heat exchanger.

Compared to a non-heated biomass digester, a heated biomass digester provides a lower residence time in the vessel, and therefore a high production.

Direct heating with steam has the disadvantage of requiring an elaborate steam-generating system (including desalination and ion exchange as water pre-treatment) and can also cause local overheating. The high cost may only be justifiable for large-scale sewage treatment facilities. The injection of hot water raises the water content of the slurry and should only be practiced if such dilution is necessary.

Indirect heating is accomplished with heat exchangers located either inside or outside of the digester, depending on the shape of the vessel, the type of substrate used, and the nature of the operating mode.
1. Floor heating systems have not served well in the past, because the accumulation of sediment gradually hampers the transfer of heat.
2. In-vessel heat exchangers are a good solution from the standpoint of heat transfer as long as they are able to withstand the mechanical stress caused by the mixer, circulating pump, etc. The larger the heat-exchange surface, the more uniformly heat distribution can be effected which is better for the biological process.
3. On-vessel heat exchangers with the heat conductors located in or on the vessel walls are inferior to in-vessel-exchangers as far as heat-transfer efficiency is concerned, since too much heat is lost to the surroundings. On the other hand, practically the entire wall area of the vessel can be used as a heat-transfer surface, and there are no obstructions in the vessel to impede the flow of slurry.
4. Ex-vessel heat exchangers offer the advantage of easy access for cleaning and maintenance.

Further components and design of the biomass digester are known to the skilled engineer.

### Reformer Section

A reformer section is arranged to receive at least a portion of the biogas stream and provide at least a first synthesis gas stream.

The first synthesis gas stream typically comprises (in % by volume)
0.5-5% methane (dry)
40-70% H₂ (dry)
10-30% CO (dry)
2-20% CO₂ (dry)

The reformer section may comprise one or more of an autothermal reforming (ATR) unit, a steam methane reforming (SMR) unit and an electrically heated steam methane reforming (e-SMR) unit, and is preferably an electrically heated steam methane reforming (e-SMR) unit. Details of an e-SMR unit that is preferably used in the reformer section are found in WO2020254121.

Additional feeds (e.g. a steam feed or oxygen-rich feed) are supplied to the reformer section, as required, depending on the type of reforming to be carried out. For instance, SMR requires a steam feed, while ATR requires a steam feed and an oxygen-rich feed.

To reduce ice formation in the downstream cryogenic separation section, the majority of water is removed from the syngas stream in the reformer section. This may be carried out by means of a cooling unit arranged to cool the syngas stream and remove water as condensate. A substantially “water free” synthesis gas stream is provided, i.e. comprising less than 100 ppm water.

### CO₂-Removal Section

A CO₂-removal section is arranged to receive a synthesis gas stream from the reformer section and provide a CO₂-rich stream and a CO₂-depleted stream. Removal of CO₂ from the synthesis gas stream prior to cryogenic separation is important; otherwise CO₂ will deposit in the cryogenic separation section.

Suitably, the CO₂-removal section comprises an amine wash unit, one or more membranes or a cryogenic CO₂-removal unit, preferably an amine wash unit. A combination of such units may also be possible.

The CO₂-removal section (40) is arranged to receive a synthesis gas stream (21) from the reformer section (20) and to provide a CO₂-rich stream (41) at least to the reformer section (20) and also to provide a CO₂-depleted stream (42) to a cryogenic separation section (30).

Optionally, the CO₂-rich stream (41) or part of it may be recycled back into the biomass digester (10).

### Cryogenic Separation Section

The cryogenic separation section is arranged to receive a substantially water-free synthesis gas stream and provide at least a CO-rich product stream.

In one embodiment, the cryogenic separation section comprises a CO cold box. A CO cold box typically comprises (in order)
i. a methane wash unit;
ii. a hydrogen stripper unit, and;
iii. a CO/CH₄ separation section.

The methane wash unit is arranged to receive a stream of syngas from the reformer section and separate it into at least an H₂-rich stream and a H₂-depleted second gas stream. In one aspect, therefore, the cryogenic separation section is also arranged to provide a H₂-rich product stream.

The hydrogen stripper unit is arranged to receive the H₂-depleted second gas stream from the methane wash unit and separate it into at least an intermediate stream and an off-gas stream. The hydrogen stripper unit uses low temperature liquid-gas separation mechanism to remove residual hydrogen in the CO—CH₄ mixture in the H₂-depleted second gas stream.

The CO/CH₄ separation section is arranged to receive the intermediate stream from said hydrogen stripper unit and separate it into at least a methane stream and a CO-rich stream. The cryogenic separation section is thus also arranged to provide a methane-rich product stream. The CO/CH₄ separation section uses low temperature liquid-gas separation mechanism to separate CO from CH₄ in the intermediate stream.

In a CO cold box, substantially pure CH₄ is produced from the CO/CH₄ separation column. This is partly used to wash the synthesis gas in the first column and by doing so the liquid methane is pumped to a pressure slightly higher than the feed pressure to the cold box. Part of this methane is however subtracted from this loop to manage the overall mass balance. This is expanded and mixed with off gas from the hydrogen stripper and then used for feed cooling.

Further information on a CO cold box using the combination of a methane wash unit, a hydrogen stripper unit, and a CO/CH₄ separation section can be found in Industrial Gases Processing, edited by H.-W. Häring, Wiley-VCH Verlag, 2008.

In another embodiment, the cryogenic separation section is also arranged to provide a CO₂-rich product stream. In this case, the cryogenic separation section comprises a cryogenic CO₂ separation section.

A cryogenic CO₂ separation section typically comprises a first cooling stage of the synthesis gas, followed by cryogenic flash separation unit to separate liquid condensate from the gas phase. Cooling for the first cooling stage may be provided by the resulting product from the cryogenic flash separation unit, potentially in the combination with other coolants. Optionally, one or more of the products from the CO₂ removal section may be expanded to some extent to make a colder process gas for this cooling stage. Cryogenic separation of CO₂ must be facilitated at elevated pressure, at least above the triple point of CO₂ to allow condensation of CO₂. A suitable pressure regime is therefore at least above the triple point of 5 bar, where increased pressure gives increased liquid yields.

Suitably, the cryogenic CO₂ separation section is operated at a temperature of from ca. −30° C. to −80° C., depending on the pressure utilised. In an embodiment of the invention, the amount of CO₂ condensed in the cryogenic separation is increased by reducing the operation temperature.

In an embodiment, the cryogenic CO₂ separation section comprises a cooling unit, followed by a flash separation unit, followed by a heating unit. In an embodiment, the cryogenic CO₂ separation section comprises a gas dryer unit. Preferably, the gas dryer unit is the first unit of the cryogenic CO₂ separation section.

### Process

The present technology also provides a process for providing a CO-rich product stream from a first biomass feed, in a plant as described herein, said process comprising the steps of:
supplying a first biomass feed to the biomass digester, and providing a biogas stream,
feeding at least a portion of the biogas stream to the reformer section, and providing a first synthesis gas stream,
feeding at least a portion of a synthesis gas stream from the reformer section to a CO₂-removal section and providing a CO₂-rich stream and a CO₂-depleted stream;
feeding the CO₂-depleted stream from the CO₂-removal section to the cryogenic separation section, and providing at least a CO-rich product stream,
wherein the CO₂-rich stream (41) is recycled back into the reformer section (20).

All details of the plant described above are equally relevant for the process of the invention, mutatis mutandis.

The present invention has been described with reference to a number of embodiments and FIGURES. However, the skilled person is able to select and combine various embodiments within the scope of the invention, which is defined by the appended claims. All documents mentioned herein are incorporated by reference.

1. A carbon monoxide plant, said plant comprising:
a first biomass feed,
a biomass digester, configured to receive the first biomass feed, and to provide a biogas stream,
a reformer section configured to receive at least a portion of the biogas stream, and to provide a first synthesis gas stream,
a CO₂-removal section configured to receive a synthesis gas stream from the reformer section, to provide a CO₂-rich stream at least to the reformer section, and to provide a CO₂-depleted stream;
a cryogenic separation section, configured to receive the CO₂-depleted stream from the CO₂-removal section and to provide at least a CO-rich product stream.

2. The plant according to claim 1, wherein the CO₂-rich stream is configured to be recycled as additional feed to the biomass digester.

3. The plant according to claim 1, wherein the cryogenic separation section is also configured to provide an H₂-rich product stream.

4. The plant according to claim 1, wherein the cryogenic separation section is also configured to provide a methane-rich product stream.

5. The plant according to claim 1, wherein the reformer section comprises an electrically-heated steam methane reforming unit.

6. The plant according to claim 1, wherein the reformer section comprises a cooling unit configured to cool the first synthesis gas stream and remove water as condensate.

7. The plant according to claim 1, wherein the CO₂-removal section comprises an amine wash unit.

8. The plant according to claim 1, wherein the CO₂-removal section comprises a cryogenic CO₂-removal unit.

9. The plant according to claim 1, wherein the CO₂-removal section comprises one or more membranes.

10. A process, using the plant according to claim 1, for providing the CO-rich product stream from the first biomass feed, said process comprising:
supplying the first biomass feed to the biomass digester, and providing the biogas stream,
feeding the at least a portion of the biogas stream to the reformer section, and providing the first synthesis gas stream,
feeding the at least a portion of a synthesis gas stream from the reformer section to the CO₂-removal section, and providing the CO₂-rich stream and the CO₂-depleted stream;
feeding the CO₂-depleted stream from the CO₂-removal section to the cryogenic separation section, and providing the at least a CO-rich product stream,
wherein the CO₂-rich stream is recycled back into the reformer section.

11. The plant according to claim 1, wherein the biomass digester is configured to operate at around 50° C. to provide the biogas stream.

12. The process according to claim 10, wherein the biomass digester operates at around 50° C. to provide the biogas stream.

13. The plant according to claim 1, wherein the cryogenic separation section is configured to operate at around −30° C. to −80° C. to provide the at least a CO-rich product stream.

14. The process according to claim 10, wherein the cryogenic separation section operates at around −30° C. to −80° C. to provide the at least a CO-rich product stream.
